(19) United States
(12) Patent Application Publication
(10) Pub. No.: US 2024/0238289 A1
(43) Pub. Date: Jul. 18, 2024

(54) METHODS AND MATERIALS FOR IMPROVING ARTERIOVENOUS FISTULA MATURATION AND MAINTAINING ARTERIOVENOUS FISTULA FUNCTIONALITY

(71) Applicant: Mayo Foundation for Medical Education and Research, Rochester, MN (US)

(72) Inventors: Karl A. Nath, Rochester, MN (US); James L. Kirkland, Rochester, MN (US); Tamar Tchkonia, Rochester, MN (US)

(73) Assignee: Mayo Foundation for Medical Education and Research, Rochester, MN (US)

(21) Appl. No.: 18/432,824

(22) Filed: Feb. 5, 2024

Related U.S. Application Data
(62) Division of application No. 16/973,723, filed on Dec. 9, 2020, now Pat. No. 11,925,640.
(60) Provisional application No. 62/688,822, filed on Jun. 22, 2018.

(51) Int. Cl. A61K 31/506; A61K 31/355; A61P 9/10

(52) U.S. Cl. CPC A61K 31/506; A61K 31/355; A61P 9/10

(57) ABSTRACT

This document provides methods and materials for improving arteriovenous fistula (AVF) maturation and/or maintaining AVF functionality. For example, methods and materials for using one or more senolytic agents to improve AVF maturation, to maintain AVF functionality, and/or to maintain the patency of an AVF are provided. Methods and materials for using one or more senolytic agents to maintain functionality and/or patency of a venous graft (e.g., a venous graft that bypasses an occluded artery) also are provided.

### CROSS-REFERENCE TO RELATED APPLICATIONS

This application is a divisional of U.S. application Ser. No. 16/973,723, filed Dec. 9, 2020, which is a National Stage application under 35 U.S.C. § 371 of International Application No. PCT/US2019/038569, having an International Filing Date of Jun. 21, 2019, which claims priority to U.S. Application Ser. No. 62/688,822, filed on Jun. 22, 2018. The disclosure of the prior application is considered part of (and is incorporated by reference in) the disclosure of this application.

### STATEMENT REGARDING FEDERAL FUNDING

This invention was made with government support under DK119167 awarded by the National Institutes of Health. The government has certain rights in the invention.

### BACKGROUND

### 1. Technical Field

This document relates to methods and materials for improving arteriovenous fistula (AVF) maturation and/or maintaining AVF functionality. For example, this document relates to using one or more senolytic agents (e.g., dasatinib, a tocotrienol, quercetin, or a combination thereof) to improve AVF maturation, to maintain AVF functionality, and/or to maintain the patency of an AVF. This document also relates to using one or more senolytic agents (e.g., dasatinib, a tocotrienol, quercetin, or a combination thereof) to maintain functionality and/or patency of a venous graft (e.g., a venous graft used to bypass an arterial occlusion).

### 2. Background Information

The formation of an AVF abruptly conjoins vascular segments that became separate and distinct early in vascular development. During embryogenesis, subsets of angioblasts in the primitive capillary plexus move apart and differentiate into what will become arteries and veins (Potente et al., Nat. Rev. Mol. Cell Biol., 18:477-494 (2017)). Vascular specialization is driven by signaling species from surrounding tissue and angioblasts themselves, the final structures of arteries and veins also shaped by their respective hemodynamic profiles (Potente et al., Nat. Rev. Mol. Cell Biol., 18:477-494 (2017)). Fully developed arteries are resilient with high intraluminal pressure, pulsatile flow, and low capacitance. Veins, conversely, are flaccid and delicate-walled, with low intraluminal pressure, low and steady flow, and high capacitance. An AVF, in a certain sense, thus reverses the course of vascular development, thereby subjecting veins to the heightened pressures and flows they were never designed to bear, and imposing pathologic shear stress on both vessels. The study of the AVF thus offers insights into vascular behavior when the vasculature is uniquely and severely stressed.

The study of the AVF also is of fundamental clinical importance as an AVF, created in superficial, upper extremity vessels, provides a therapeutic device: namely, a high-flow, accessible, venous segment for hemodialysis for endstage renal disease (ESRD). Indeed, the AVF, compared with other accesses, is the most favored, promoted as it is by the “Fistula First” initiative (Mehrotra et al., J. Am. Soc. Nephrol., 28:395-397 (2017); Nath et al., Clin. J. Am. Soc. Nephrol., 12:2053-2055 (2017); Riella et al., Nat. Rev. Nephrol., 9:348-357 (2013); Roy-Chaudhury et al., J. Am. Soc. Nephrol., 17:1112-1127 (2006); and Viecelli et al., Semin. Dial., 31:244-257 (2018)).

To support intermittent hemodialysis, the AVF must undergo “maturation.” Maturation requires markedly increased blood flow as the artery dilates and both vessels undergo luminal expansion and outward remodeling. Outward remodeling also toughens the vein, allowing it to be repeatedly punctured for dialysis (Nath et al., J. Am. Soc. Nephrol., 27:3508-3510 (2016); Riella et al., Nat. Rev. Nephrol., 9:348-357 (2013); and Roy-Chaudhury et al., J. Am. Soc. Nephrol., 17:1112-1127 (2006)). Additionally, maturation and sustained function require suppression of inward remodeling (neointimal hyperplasia and thrombogenesis) (Nath et al., J. Am. Soc. Nephrol., 27:3508-3510 (2016); Riella et al., Nat. Rev. Nephrol., 9:348-357 (2013); and Roy-Chaudhury et al., J. Am. Soc. Nephrol., 17:1112-1127 (2006)).

Clinical AVF outcomes, however, are truly grim: 50% of created AVFs may never mature; 30% require instrumentation to mature; and the median survival for AVFs is 7.5 months, which increases to 62 months when primary AVF failure is excluded (Lok et al., Clin. J. Am. Soc. Nephrol., 8:810-818 (2013)). Even after maturation, 15% will be nonfunctional after 1 year, and 25% after 2 years (Roy-Chaudhury et al., J. Am. Soc. Nephrol., 17:1112-1127 (2006)). Dysfunction of AVFs and other accesses markedly contributes to morbidity, mortality, and hospitalization rates (˜25%) in patients with ESRD, imposing healthcare costs that may exceed $2.5 billion/year (USRDS 2009 Annual Data Report: Atlas of Chronic Kidney Disease and End-Stage Renal Disease in the United States. Bethesda (MD): U.S. Renal Data System, National Institutes of Health, National Institute of Diabetes and Digestive and Kidney Diseases, 2009).

There is no specific therapy that promotes maturation and functionality of human AVFs. Of all the unmet needs in ESRD, this is among the most pressing (Nath et al., Clin. J. Am. Soc. Nephrol., 12:2053-2055 (2017); Roy-Chaudhury et al., J. Am. Soc. Nephrol., 17:1112-1127 (2006); and Viecelli et al., Semin. Dial., 31:244-257 (2018)). Strategies targeted to this need largely rely on acceptable rodent AVF models to understand the basis for AVF failure. Studies in rodent and human AVFs have revealed an induction of potentially vasculopathic species, but, thus far, the underlying mechanism that underpins vasculopathic responses remains elusive.

### SUMMARY

This document provides methods and materials for improving AVF maturation and/or maintaining AVF functionality. For example, this document provides methods and materials for using one or more senolytic agents (e.g., dasatinib, a tocotrienol, quercetin, or a combination thereof) to improve AVF maturation, to maintain AVF functionality, and/or to maintain the patency of an AVF. This document also provides methods and materials for using one or more senolytic agents (e.g., dasatinib, a tocotrienol, quercetin, or a combination thereof) to maintain functionality and/or patency of a venous graft (e.g., a venous graft used to bypass an arterial occlusion).

As described herein, administering a composition comprising one or more senolytic agents to a mammal (e.g., a human) having a surgically created AVF can promote maturation of the AVF such that it can be used as an effective hemodialysis vascular access. In some cases, a composition comprising one or more senolytic agents can be administered locally to an AVF (e.g., a surgically created AVF) to promote maturation of the AVF. Having the ability to promote maturation of an AVF as described herein can allow clinicians and patients to use effective hemodialysis vascular access points that would not otherwise be available. Having the ability to promote maturation of an AVF as described herein also can allow clinicians and patients to achieve efficient hemodialysis that would not otherwise be available.

As also described herein, administering a composition comprising one or more senolytic agents to a mammal (e.g., a human) having a surgically created AVF can maintain functionality and/or patency of the AVF such that it can continue to be used as an effective hemodialysis vascular access. In some cases, a composition comprising one or more senolytic agents can be administered locally to an AVF (e.g., a surgically created AVF) to maintain functionality and/or patency of the AVF. Having the ability to maintain functionality and/or patency of an AVF as described herein can allow clinicians and patients to use effective hemodialysis vascular access points for extended periods of time.

In addition, as described herein, administering a composition comprising one or more senolytic agents to a mammal (e.g., a human) having a venous graft (e.g., a venous graft attached to an artery such as a venous graft for bypassing an occluded artery) can maintain functionality and/or patency of the venous graft. In some cases, a composition comprising one or more senolytic agents can be administered locally to a venous graft to maintain functionality and/or patency of the venous graft. Having the ability to maintain functionality and/or patency of a venous graft as described herein can allow clinicians and patients to use functional venous grafts for extended periods of time.

In general, one aspect of this document features a method for promoting arteriovenous fistula maturation within a mammal. The method comprises (or consists essentially of or consist of) administering a composition comprising a senolytic agent to the mammal, wherein the arteriovenous fistula matures into an effective vascular access point for hemodialysis. The mammal can be a human. The composition can comprise dasatinib, a tocotrienol, quercetin, or a combination thereof. The composition can comprise dasatinib and quercetin. The composition can comprise a tocotrienol and quercetin. The administration can comprise a local administration to the arteriovenous fistula. In some cases, the senolytic agent can be the sole active ingredient of the composition to promote arteriovenous fistula maturation within the mammal.

In another aspect, this document features a method for maintaining functionality of an arteriovenous fistula within a mammal. The method comprises (or consists essentially of or consist of) administering a composition comprising a senolytic agent to the mammal, wherein the arteriovenous fistula remains functional as effective vascular access point for hemodialysis following the administering step. The mammal can be a human. The composition can comprise dasatinib, a tocotrienol, quercetin, or a combination thereof. The composition can comprise dasatinib and quercetin. The composition can comprise a tocotrienol and quercetin. The administration can comprise a local administration to the arteriovenous fistula. In some cases, the senolytic agent can be the sole active ingredient of the composition to maintain functionality of the arteriovenous fistula within the mammal.

In another aspect, this document features a method for maintaining patency of an arteriovenous fistula within a mammal. The method comprises (or consists essentially of or consist of) administering a composition comprising a senolytic agent to the mammal, wherein the arteriovenous fistula remains open following the administering step. The mammal can be a human. The composition can comprise dasatinib, a tocotrienol, quercetin, or a combination thereof. The composition can comprise dasatinib and quercetin. The composition can comprise a tocotrienol and quercetin. The administration can comprise a local administration to the arteriovenous fistula. In some cases, the senolytic agent can be the sole active ingredient of the composition to maintain patency of the arteriovenous fistula within the mammal.

In another aspect, this document features a method for maintaining functionality of venous graft within a mammal. The method comprises (or consists essentially of or consist of) administering a composition comprising a senolytic agent to the mammal, wherein the venous graft remains functional as effective graft following the administering step. The mammal can be a human. The composition can comprise dasatinib, a tocotrienol, quercetin, or a combination thereof. The composition can comprise dasatinib and quercetin. The composition can comprise a tocotrienol and quercetin. The administration can comprise a local administration to the venous graft. The venous graft can be a venous graft that bypasses an occluded artery within the mammal. The venous graft can be attached to a coronary artery. The mammal can have an ischemic heart disease. The venous graft can be attached to a carotid artery. The mammal can have a cerebrovascular disease. The venous graft can be attached to a femoral or popliteal artery. The mammal can have a peripheral arterial disease. The venous graft can be attached to a renal artery. The mammal can have a renovascular disease. The venous graft can be attached to a mesenteric artery. The mammal can have a mesenteric arterial disease or abdominal angina. In some cases, the senolytic agent can be the sole active ingredient of the composition to maintain functionality of the venous graft within the mammal.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and drawings, and from the claims.

### DESCRIPTION OF DRAWINGS

FIGS. 1A-E. Stressors in the vein in the AVF-CKD model. A. 3D Color Doppler ultrasound of the AVF in the AVF-CKD group. Red and blue colors indicate cranial and caudal blood flow in the AVF artery and vein, respectively. Oscillatory flow at the anastomosis is revealed by the yellow mosaic pattern, and a mixture of caudal (blue) and cranial-directed (red) flow is seen in the dilated AVF vein. B. Western analysis of PCNA and β-catenin expression in sham veins and in the vein of the AVF-CKD model, and normalized expression at one week (*P<0.05). C. IL-1a, IL-6, and MCP-1 mRNA expression in sham veins and in the vein of the AVF-CKD model at one week; n=9 and n=10 for sham and AVF-CKD groups, respectively, *P<0.01. D. pO₂ in blood from sham veins and from the vein of the AVF-CKD model after AVF/sham surgery; n=6 in each group, *P<0.05. E. Heat map of RNA-seq analysis of sham veins and the vein of the AVF-CKD model at one week. The map is comprised of 2,427 genes with a false discovery rate below 0.05 and an absolute log 2 fold change of 2 or greater. Green to red coloring was calculated from each sample's RPKM value for each gene and then transformed to z-scores. Significant transcriptomic landscape differences distinguish each group; unsupervised clustering with the MeV software perfectly separated the sham vein samples from the AVF-CKD samples.

FIGS. 2A-C. Venous senescence markers in the AVF-CKD model. A. Expression of genes encoding for p16Ink4a (Cdkn2a) and p21Cip1 (Cdkn1a) in sham veins and in the vein of the AVF-CKD model, measured by quantitative real-time RT-PCR; n=9 and n=10 for sham and AVF-CKD groups, respectively, *P<0.0001. B. Western analysis of p53, p21Cip1, and p16Ink4a protein expression in sham veins and in the vein of the AVF-CKD model, and normalized expression (*P<0.001). C. miRNA expression of miR-21 (left) and miR-92a (right) in sham veins and in the vein of the AVF-CKD model; n=8 and n=10 for sham and AVF-CKD groups, respectively *P<0.001.

FIGS. 3A-C. Telomere erosion and SA-β-Gal activity/staining in the vein in AVF-CKD. A. Telomere erosion in the AVF-CKD vein as shown by a disproportionate loss of gene expression in the telomeric regions, defined as 10 MBs from the start or end of each autosomal chromosome. Differentially expressed genes were defined by having a false discovery rate equal to or lower than 0.05 when comparing the AVF-CKD group to the sham group. A total of 897 genes in the telomeric regions have decreased expression with a log 2 fold change less than 0, 66 of which have a log 2 fold change less than −4. B. SA-β-Gal activity in sham vein and the vein of the AVF-CKD model at one week; n=5 in each group, *P<0.0001. C. SA-β-Gal activity in frozen sections from sham vein and the vein of the AVF-CKD model at one week. Cells exhibiting increased SA-β-Gal activity exhibit the blue color. The sham group (left) shows no staining, whereas the AVF-CKD model shows blue staining in the endothelium and media.

FIGS. 4A-C. p16Ink4a expression in arteries in AVF-CKD, in AVF without CKD, and in the vein in AVF-CKD following administration of senolytics. p16Ink4a protein expression at one week in sham arteries and the artery of the AVF-CKD model (A), in sham veins and the vein of the AVF in mice with normal kidneys (B), and in the vein of the AVF-CKD model administered either vehicle or dasatinib and quercetin (D/Q, C). Normalized expression is below each Western blot. *P<0.001 for A and B, and *P<0.05 for C.

FIGS. 5A-B. Senescence-associated β-Galactosidase (SA-β-Gal) activity in the vein and artery of the rat AVF-CKD model. Activity in veins (A) and arteries (B) from sham and AVF-CKD rats was assessed at 1 week after AVF creation. n=6 and n=7 in the sham and AVF-CKD groups, respectively. *P<0.005.

FIGS. 6A-B. Senescence-associated β-Galactosidase (SA-β-Gal) activity in the vein and artery of the rat AVF-CKD model. Activity in veins (A) and arteries (B) from sham and AVF-CKD rats was assessed at 2 weeks after AVF creation. n=5 and n=6 in the sham and AVF-CKD groups, respectively. *P<0.005.

FIGS. 7A-B. p21Cip1 expression in the vein of the rat AVF-CKD model. p21Cip1 mRNA (Cdknla, A) and protein (B) expression was assessed in veins from sham and AVF-CKD rats was assessed at 1 week after AVF creation. n=7 and n=9 in the sham and AVF-CKD groups, respectively in A, n=6 and n=7 in the sham and AVF-CKD groups, respectively in B and. *P<0.005.

FIG. 8. p16Ink4a expression in the vein of the rat AVF-CKD model. p16Ink4a mRNA (Cdkn2a) expression was assessed in veins from sham and AVF-CKD rats at 1 week after AVF creation. n=7 and n=9 in the sham and AVF-CKD groups, respectively. *P<0.005.

### DETAILED DESCRIPTION

This document provides methods and materials for improving AVF maturation and/or maintaining AVF functionality. For example, this document provides methods and materials for using one or more senolytic agents (e.g., dasatinib, a tocotrienol, quercetin, or a combination thereof) to improve AVF maturation, to maintain AVF functionality, and/or to maintain the patency of an AVF within a mammal (e.g., a human). This document also provides methods and materials for using one or more senolytic agents (e.g., dasatinib, a tocotrienol, quercetin, or a combination thereof) to maintain functionality and/or patency of a venous graft (e.g., a venous graft used to bypass an arterial occlusion) within a mammal (e.g., a human).

Examples of mammals that can be treated as described herein include, without limitation, humans, monkeys, dogs, cats, cows, horses, pigs, rats, and mice. In some cases, the mammal can be a mammal (e.g., a human) that requires hemodialysis and has an arteriovenous fistula. In some cases, the mammal can be a mammal (e.g., a human) that has a venous graft.

Arteries are elastic and resilient with high intraluminal pressure, pulsatile flow, and low capacitance. Veins are flaccid and delicate-walled, lack tensile strength, and are characterized by low intraluminal pressure, low and steady flow, and high capacitance. Venous blood contains oxygen at a relatively low partial pressure of oxygen, whereas the partial pressure of oxygen in arterial blood is markedly higher. Connecting a vein to the side of an artery, as is done when a dialysis AVF is created, subjects the vein to greatly increased blood pressures, blood flow rates, and blood oxygenation, all of which the vein was never designed to bear. As described herein, this profound hemodynamic and oxygenation stress can cause accelerated senescence of the vein, and, ultimately, failure of the vein to mature and/or AVF failure. The methods and material provided herein can be used to promote maturation of an AVF within a mammal and/or to maintain the functionality and/or patency of an AVF within a mammal. For example, a composition containing one or more senolytic agents can be administered to a mammal (e.g., a human) having an AVF to promote maturation of the AVF within the mammal and/or to maintain the functionality and/or patency of the AVF within the mammal.

Veins also are anastomosed to arteries when there is an occlusion in the artery that impedes blood flow to a relevant organ or tissue. The proximal end of this venous graft can be anastomosed “upstream” of the arterial occlusion, and the distal end of this venous graft can be anastomosed “downstream” of the arterial occlusion. In this way, the arterial occlusion can be entirely bypassed by the venous graft. These venous grafts can be subjected to the markedly increased blood pressures, blood flow, and blood oxygenation of the arterial circulation. Such hemodynamic and oxygenation stress can cause senescence in these venous grafts used to bypass diseased arteries, causing these venous grafts to fail. Venous grafts are widely used to bypass arterial occlusions in diseased arteries as broadly occurs in atherosclerotic cardiovascular disease such cardiovascular disease, which is a leading cause of death in the United States and other developed countries. The methods and material provided herein can be used to maintain the functionality and/or patency of a venous graft within a mammal. For example, a composition containing one or more senolytic agents can be administered to a mammal (e.g., a human) having a venous graft to maintain the functionality and/or patency of the venous graft within the mammal.

Examples of venous grafts used to bypass an occluded artery that can be treated with a composition containing one or more senolytic agents as described herein to maintain functionality and/or patency of the venous graft include, without limitation, venous grafts used in coronary arteries in mammals with, for example, ischemic heart disease, venous grafts used in carotid arteries in mammals with, for example, cerebrovascular disease, venous grafts used in femoral or popliteal arteries in mammals with, for example, peripheral arterial disease, venous grafts used in renal arteries in mammals with, for example, renovascular disease, and venous grafts used in mesenteric arteries in mammals with, for example, mesenteric arterial disease (e.g., abdominal angina).

Examples of senolytic agents that can be used as described herein include, without limitation, dasatinib, a tocotrienol, quercetin, fisetin, luteolin, navitoclax, A1331852 (CAS Number 1430844-80-6; 6-[8-[(2-benzothiazolylamino)carbonyl]-3,4-dihydro-2(1H)-isoquinolinyl]-3-[5-methyl-1-(tricyclo[3.3.1.13.7]dec-1-ylmethyl)-1H-pyrazol-4-yl]-2-pyridinecarboxylic acid), A1155463 (CAS Number 1235034-55-5; 2-[8-[(2-Benzothiazolylamino)carbonyl]-3,4-dihydro-2(1H)-isoquinolinyl]-5-[3-[4-[3-(dimethylamino)-1-propyn-1-yl]-2-fluorophenoxy]propyl]-4-thiazolecarboxylic acid), 17-AAG (Tanespimycin), geldanamycin, piperlongumine, 17-DMAG (Alvespimycin), and panobinostat.

A composition for treating an AVF and/or venous graft as described herein can include one senolytic agent or more than one senolytic agent (e.g., two, three, four, five, or more senolytic agents). For example, a composition can be designed to include dasatinib and quercetin and can be used promote AVF maturation in a mammal. In some cases, two, three, four, or five different senolytic agents can be administered in combination or sequentially to a mammal with an AVF and/or a venous graft.

In some cases, a senolytic agent can be chemically converted from its free base form to a pharmaceutically acceptable salt by reacting the free base with an equivalent amount of an acid that forms a non-toxic salt, which can be used to treat an AVF and/or venous graft as described herein or to form a composition that can be used to treat an AVF and/or venous graft as described herein. Such acids can be either inorganic or organic including, without limitation, hydrochloric acid, hydrobromic acid, fumaric acid, maleic acid, succinic acid, sulfuric acid, phosphoric acid, tartaric acid, acetic acid, citric acid, and oxalic acid. In some cases, a senolytic agent (or a pharmaceutically acceptable salt thereof) provided herein can be administered to a mammal by itself or in combination with a carrier. Such carriers include, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. In some cases, preservatives, flavorings, and other additives such as, for example, antimicrobials, anti-oxidants, chelating agents, inert gases, and the like can be present. It will be appreciated that a senolytic agent (or a pharmaceutically acceptable salt thereof) provided herein that is to be administered to a mammal can contain zero, one, or more than one pharmaceutically acceptable carriers.

A senolytic agent or a composition containing one or more senolytic agents provided herein can be administered to any part of a mammal's body. For example, a senolytic agent or a composition containing one or more senolytic agents provided herein can be administered directly to an AVF or venous graft of a mammal. In some cases, a senolytic agent or a composition containing one or more senolytic agents provided herein can be administered via an adventitial administration. In some cases, a senolytic agent or a composition containing one or more senolytic agents provided herein can be administered intravenously, subcutaneously, intraperitoneal, or orally.

When treating a mammal (e.g., a human) having an AVF, an effective amount of a senolytic agent or a composition containing one or more senolytic agents provided herein can be any amount that, when administered to the mammal having an AVF, promotes AVF maturation and/or maintains the functionality and/or patency of the AVF as described herein without inducing significant toxicity in the mammal. When treating a mammal (e.g., a human) having a venous graft, an effective amount of a senolytic agent or a composition containing one or more senolytic agents provided herein can be any amount that, when administered to the mammal having a venous graft, maintains the functionality and/or patency of the venous graft as described herein without inducing significant toxicity in the mammal. Such amounts can be determined using the methods and materials provided herein. Some compounds may have a relatively broad concentration range that is effective, while others may have a relatively narrow effective concentration range. In addition, the effective amount can vary depending upon the specific mammal or the specific condition of an AVF or venous graft. Such effective amounts can be determined for individual compounds using available or ascertainable information involving equilibrium dissociation constants, mammal toxicity concentrations, and bioavailability. For example, non-toxic compounds typically can be directly or indirectly administered to a mammal in any amount that induces a desired result in that mammal.

Using the information provided herein, such effective amounts also can be determined in vitro or in vivo. For example, a mammal (e.g., a human) can receive direct administration of a senolytic agent provided herein in an amount to achieve a desired result. If the patient fails to respond, then the amount can be increased by, for example, two fold. After receiving this higher concentration, the patient can be monitored for both responsiveness to the treatment and toxicity symptoms, as well as blood levels of the drug, and adjustments made accordingly.

Various factors can influence the actual effective amount used for a particular application. For example, the frequency of administration, duration of treatment, rate of metabolism of the senolytic agent, combination of other compounds, and site of administration may require an increase or decrease in the actual effective amount administered.

The frequency of administration can be any frequency that induces a desired result within a mammal without producing significant toxicity to the mammal. For example, the frequency of administration can be from about twice a day to about once a month, or more specifically, from about once a week to about once a month (e.g., orally or topically, for example, as a cream, ointment, emulsion, or suspension over the site of the AVF, or by injection into the fistula or systemically). In addition, the frequency of administration can remain constant or can be variable during the duration of treatment. As with the effective amount, various factors can influence the actual frequency of administration used for a particular application. For example, the effective amount, duration of treatment, rate of metabolism of the senolytic agent, combination of other compounds, and site of administration may require an increase or decrease in administration frequency.

An effective duration for administration of a compound provided herein can be any duration that induces a desired result within a mammal without producing significant toxicity to the mammal. Thus, the effective duration can vary from several days to several weeks, months, or years. In general, the effective duration for promoting AVF maturation into an effective hemodialysis vascular access point in a mammal can range in duration from one day to one, two, three, four, or five months. In some cases, the effective duration for maintaining the functionality and/or patency of an AVF in a mammal can range in duration from one day, one week, or one month to one, two, three, four, five, or more years. In some cases, the effective duration for maintaining the functionality and/or patency of a venous graft in a mammal can range in duration from one day, one week, or one month to one, two, three, four, five, or more years. Multiple factors can influence the actual effective duration used for a particular treatment regimen. For example, an effective duration can vary with the frequency of compound administration, effective compound amount, combination of multiple compounds, and site of administration.

The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

The invention will be further described in the following examples, which do not limit the scope of the invention described in the claims.

### EXAMPLES

### Example 1—Dialysis Fistula Exhibits a Senescence Phenotype in Mice

### Methods and Materials

The AVF-CKD model was created in male C57BL/6J mice as described elsewhere in which an AVF/sham AVF was created in mice in which CKD/sham CKD was previously imposed (Kang et al., Am. J. Physiol. Renal Physiol., 310: F466-476 (2016)). At one week after AVF/sham AVF, the vasculature was harvested for assorted analyses and assessment of senescence-associated beta galactosidase (SA-β-Gal) activity. At two weeks, ultrasound studies were performed. In selected studies, pO₂ was measured by an i-Stat analyzer (Abbott Point of Care, Princeton, NJ) in venous blood samples drawn immediately after AVF/sham AVF. In other studies, mice with AVF-CKD were treated with the senolytic agent combination (dasatinib, 5 mg/kg, and quercetin, 50 mg/kg) or vehicle via gavage on days 4 and 5 after AVF creation (Schafer et al., Nat. Commun., 8:14532 (2017); and Zhu et al., Aging Cell, 14:644-658 (2015)), and p16Ink4a protein expression was assessed at 7 days.

### Vascular Ultrasound

Sonography was performed using a high-resolution imaging (Vevo2100 with a MS400 30-MHz transducer, Vevo3100 with a MX400 38-MHz transducer, FUJIFILM VisualSonics Inc., Toronto, Canada) (Wang et al., Arterioscler. Thromb. Vasc. Biol., 35:1401-1412 (2015)). Blood flow dynamics was three-dimensionally (3D) reconstructed from a serial 2D color Doppler at 32-μm intervals (Wang et al., Arterioscler. Thromb. Vasc. Biol., 35:1401-1412 (2015)).

### Gene Expression by RNA-Seq

Total RNA was extracted as described elsewhere (Kang et al., Am. J. Physiol. Renal Physiol., 310:F466-476 (2016)). RNA-Sequencing samples were processed through Mayo Clinic's MAP-RSeq V2 software (Kalari et al., BMC Bioinformatics, 15:224 (2014)). This was a comprehensive RNA-Sequencing workflow for Illumina paired end reads. The reference genome and transcriptome used was Ensembl's Mus musculus GRCm38.79. Quality control metrics from RSeqQC were evaluated to insure that raw expression values from each sample were reliable and could be collectively used for differential expression analysis (Wang et al., Bioinformatics, 28:2184-2185 (2012)). Genes with an average of 25 or more reads were kept for performing differential expression analysis. The R package (edgeR) was used to identify which genes were statistically differentially expressed from the group comparisons (Robinson et al., Bioinformatics, 26:139-140 (2010)). For each comparison, unsupervised clustering was implemented with the MeV (Howe et al., Bioinformatics, 27:3209-3210 (2011)) application on genes with a false discovery rate below 0.05 and with an absolute log 2 fold change of 2 or greater. MeV was additionally used to visualize these findings through heat maps.

mRNA and microRNA Expression

mRNA expression was performed as described (Kang et al., Am. J. Physiol. Renal Physiol., 310:F466-476 (2016); and Kang et al., Am. J. Physiol. Heart Circ. Physiol., 308:H1402-1413 (2015)) using quantitative real-time RT-PCR. microRNA (miRNA) expression was determined in total RNA using a mirVana miRNA isolation kit (Thermo Fisher Scientific, Waltham, MA). Quantitation was performed using Taqman miRNA assays (Applied Biosystems, Foster City, CA).

### Western Blot Analysis

Vascular protein extracts were prepared as described elsewhere (Kang et al., Am. J. Physiol. Renal Physiol., 310:F466-476 (2016)). Proteins were separated on TGX Stain-Free gels (BioRad, Hercules, CA), visualized by UV light-activation, and transferred to PVDF membranes. Primary antibodies included: p16Ink4a, p21Cip1 (Abcam, Cambridge, MA), p53 (R & D systems. Minneapolis, MN), PCNA (Cell Signaling Technology, Danvers, MA), and β-catenin (BD Biosciences, Franklin Lakes, NJ). Densitometric quantitation of protein expression was normalized to total protein visualized on the PVDF membranes (Gel Doc XR+ imager, BioRad).

### SA-β-Gal Activity

This was quantified using a fluorometric assay kit (Enzo Life Sciences, Farmingdale, NY). Staining for SA-β-Gal activity in frozen sections was performed as described elsewhere (Ogrodnik et al., Nat. Commun., 8:15691 (2017)).

### Statistics

Data were expressed as means±SE and considered statistically significant for P<0.05. The Student's t-test was used for parametric data and the Mann-Whitney U-test was employed for nonparametric data.

### Results

### The AVF Exhibits Diverse Stressors and Altered Gene Expression

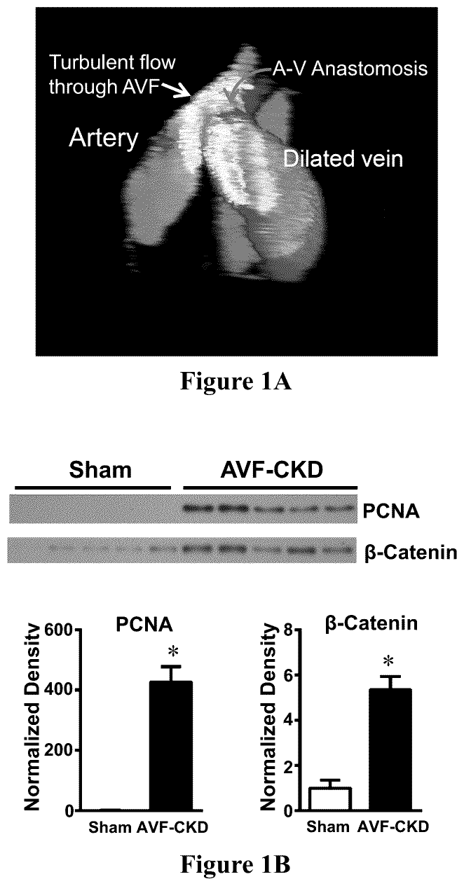

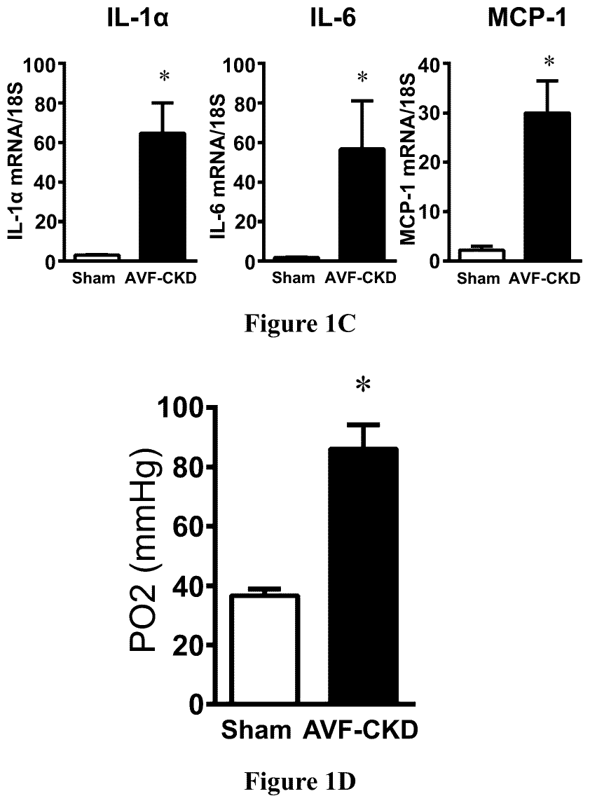

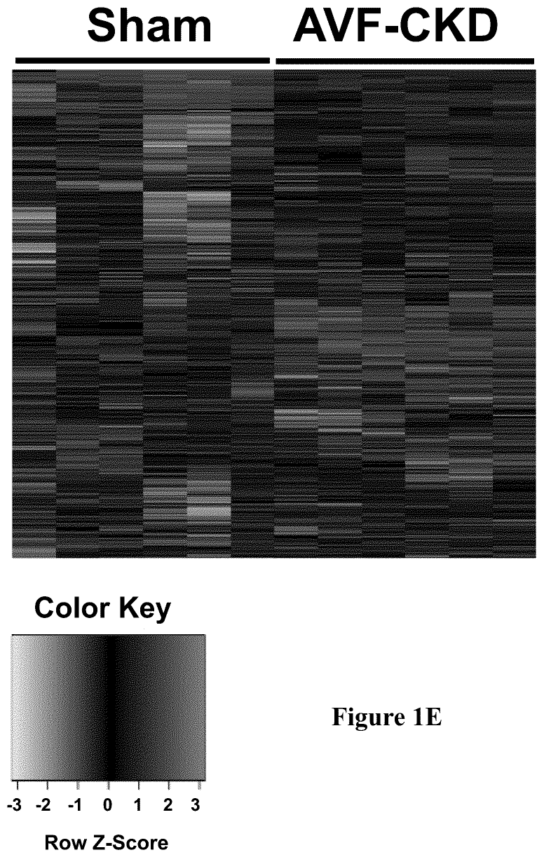

As stress induces vasculopathic responses, whether relevant forms of stress exist in the vein of the AVF was examined. 3D Doppler ultrasound demonstrated the presence of pathologic oscillatory shear stress and turbulent flow (FIG. 1A). Mitogenic and inflammatory stress were also present. Compared with the sham group, the vein of AVF-CKD group exhibited markedly upregulated proliferation markers (PCNA), proliferative signaling species (β-catenin) (FIG. 1B), and pro-inflammatory cytokines (FIG. 1C). Whether reoxygenation stress occurred in the AVF also was considered. Intact veins are normally exposed to a low pO₂ to which their metabolism is fully geared. AVF creation immediately exposed the vein to a markedly increased pO₂, thereby imposing “hypoxia-reoxygenation” stress on the AVF-CKD (FIG. 1D).

Such stressors were accompanied by altered gene expression in the AVF, as demonstrated by RNA-Seq analyses. Some 20,000 genes were detected, with approximately 10,000 being different in the vein of the AVF-CKD group, including the upregulation of proinflammatory and proliferative genes. The heat map revealed clearly visible transcriptomic landscape differences, thereby distinguishing the veins in the AVF-CKD and Sham groups (FIG. 1E).

### AVF Exhibits a Senescence Phenotype

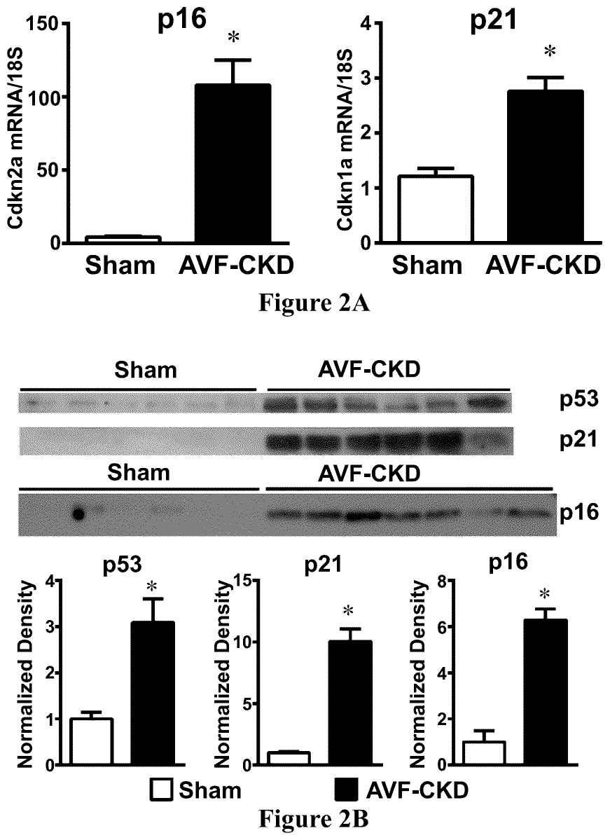

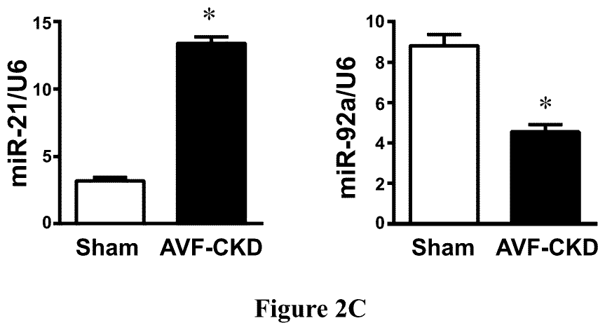

Stress, especially when diverse, may induce premature senescence (Kirkland et al., EBioMedicine, 21:21-28 (2017); Kirkland et al., J. Am. Geriatr. Soc., 65:2297-2301 (2017); and Tchkonia et al., J. Clin. Invest., 123:966-972 (2013)). Whether a senescence phenotype exists in the AVF was examined, beginning with the senescence drivers, p16Ink4a and p21Cip1. Cdkn2a (reflecting mainly p16) and Cdkn1a (reflecting mainly p21) mRNAs were induced in the vein of the AVF. This was accompanied by increased p16Ink4a and p21Cip1 protein expression (FIGS. 2A and 2B). In addition, induction of p53 protein (upstream of p21Cip1 in the senescence pathway) occurred (FIG. 2B). The AVF-CKD vein exhibited changes for certain senescence-associated miRNAs (Olivieri et al., Ageing Res. Rev., 12:1056-1068 (2013); and Hazra et al., Exp. Gerontol., 83:165-170 (2016)). In particular, the AVF-CKD vein exhibited, congruently, increased expression of miR-21 and decreased expression of miR-92a (FIG. 2C).

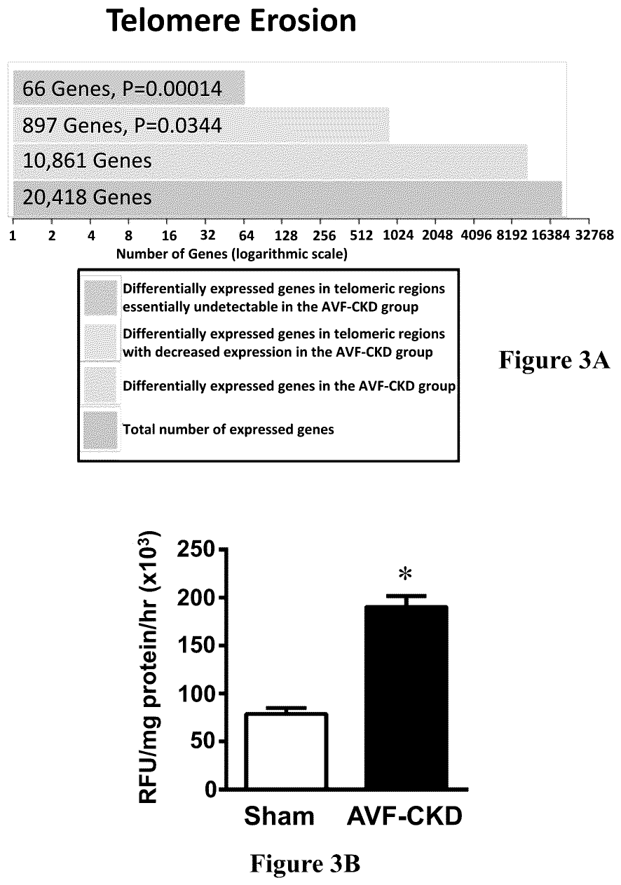

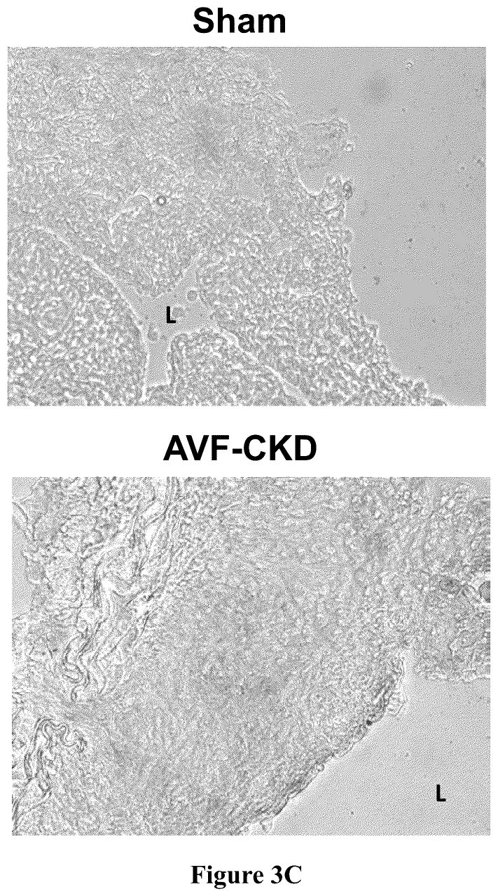

Additional RNA-Seq analyses focused on telomeric regions. Compared with the Sham group, the vein in the AVF-CKD group showed some 900 genes with significantly decreased expression and 66 genes that were essentially undetectable (FIG. 3A). Telomere erosion, a senescence hallmark, thus existed in the vein in the AVF-CKD group. Also present at one week was increased SA-β-Gal activity (FIG. 3B). Studies at one week revealed increased SA-β-Gal (blue) staining in the vein in the AVF-CKD group, with focal cellular staining in the endothelium and tunica media; the control vein was devoid of blue staining (FIG. 3C).

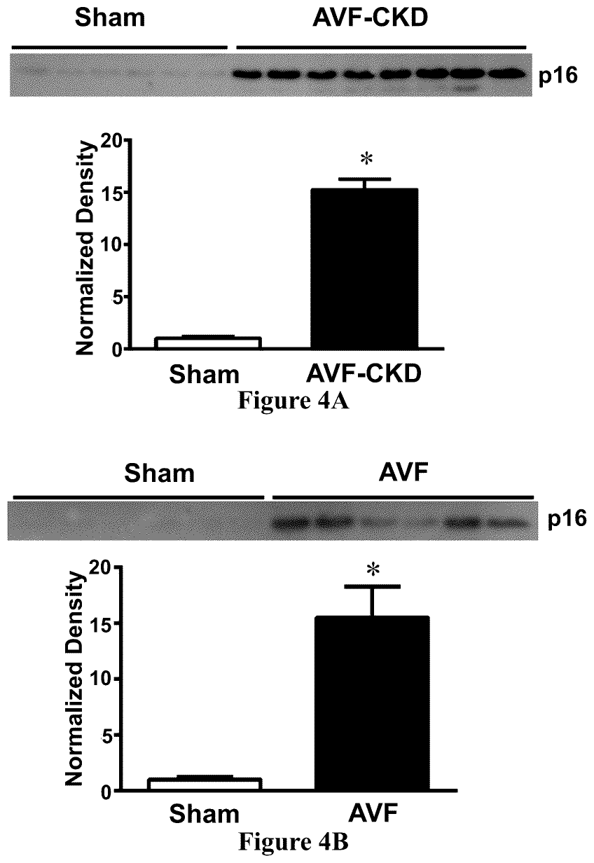

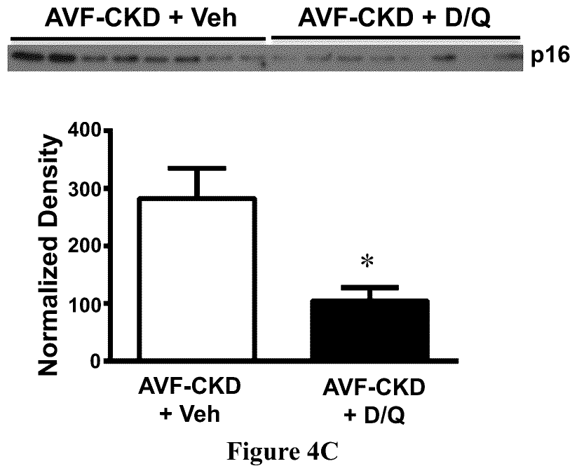

Senescence changes also occurred in the AVF artery as revealed by striking p16Ink4a induction in the artery of the AVF-CKD group (FIG. 4A). The AVF itself, in presence of normal kidney function, evinced p16Ink4a upregulation (FIG. 4B). Finally, the short-term effect of senolytics (dasatinib and quercetin) was examined. Dasatinib and quercetin significantly decreased expression of p16Ink4a at 1 week in the vein in the AVF-CKD group (FIG. 4C).

These results demonstrate that senolytic agents (e.g., locally applied senolytic agents or senolytic agents administered orally, for example, by gavage) can be used to improve arteriovenous fistula maturation, to improve arteriovenous fistula functionality, and to prevent failure of arteriovenous fistulas. These results also demonstrate that senolytic agents can be used to prevent failure of venous grafts, such as venous grafts used to bypass occluded arteries.

### Example 2—Dialysis Fistula Exhibits a Senescence Phenotype in Rats

This Examples describes studies in a rat model of the fistula (AVF-CKD) to determine whether the senescence phenotype could be exhibited in multiple species (see Example 1 for studies using a mouse model of the fistula).

### Methods and Materials

Rats were first subjected to subtotal nephrectomy, which creates chronic kidney disease (CKD). The rats were anesthetized with pentobarbital (60 mg/kg, IP), and a midline abdominal incision was performed. The gut was gently retracted, thereby exposing the renal pedicles. The right kidney was surgically removed following ligation of the right pedicle with sterile 6-0 silk suture. The arterial vasculature of the left kidney underwent selective segmental ligation with sterile 6-0 silk suture to achieve the desired degree of infarction. The abdominal wall was sutured. In a similar fashion, a sham surgery was performed consisting of the abdominal incision, retraction of the gut, and closure of the abdominal incision. One week after the subtotal nephrectomy or sham surgery, the rats were subjected to AVF surgery. After anesthesia with pentobarbital (60 mg/kg, IP), the rats were placed on a heated surface to maintain body temperature at 37° C. The femoral vasculature was exposed by an incision along the entire length of the inguinal fold and subsequent retraction of the abdominal musculature medially and the inguinal fat pad laterally. The remainder of the procedure was performed under a dissecting microscope at 30× magnification. Using careful dissection, the femoral artery and vein were freed from surrounding fascia and the femoral nerve. Branching vasculature from the femoral artery and vein was ligated with sterile 6-0 silk suture and divided. The femoral vein was then clamped with a non-traumatic vein clip at the proximal end of its exposure, ligated at the distal end of its exposure, and subsequently transected just proximally to the ligation at a 45 degree angle. Non-traumatic aneurysm clips were then placed on the artery at the most proximal and distal points of the exposure, and a small longitudinal incision was made in the artery where the anastomosis was made using a microsurgical knife. Both vessels were rinsed with heparinized saline, and the transected end of the vein was attached to the opening in the adjacent artery with eight equally-spaced interrupted sutures using 10-0 monofilament nylon suture. All clips were then removed, and arterial flow into the femoral vein confirmed. The inguinal incision was then closed with sterile 6-0 vicryl absorbable suture in two separate layers (muscle and skin), using a continuous pattern for each layer. In a similar fashion, a sham surgery was performed on rats, consisting of the inguinal incision, dissection of the vasculature, clamping of the femoral artery and vein, and closure of the inguinal incision. The rats were then allowed to recover from anesthesia.

Determination of β-galactosidase (SA-β-Gal) activity and expression of p21Cip1 and p16Ink4a was performed as described for the mouse.

### Results

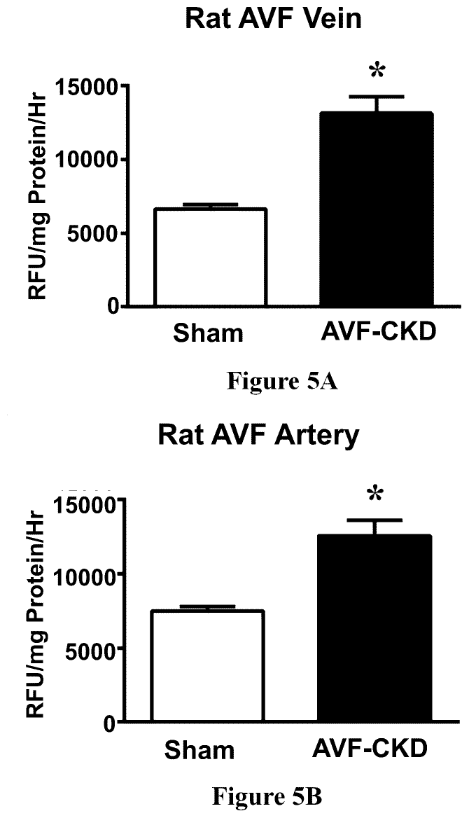

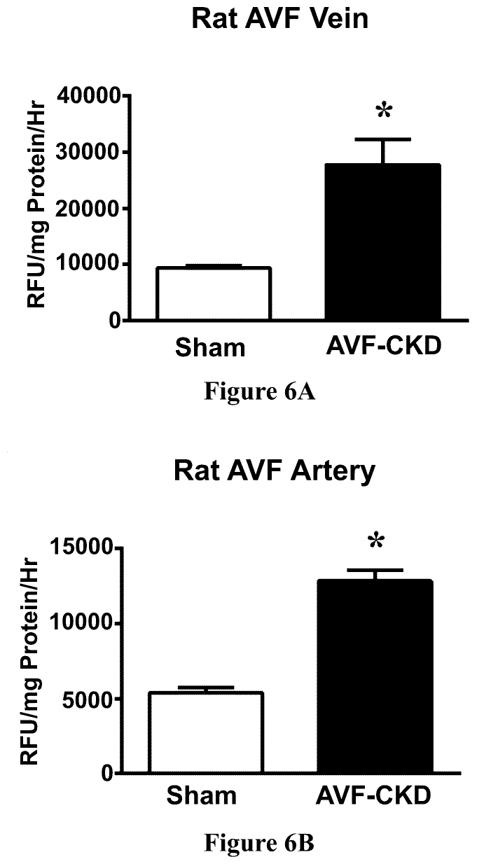

β-galactosidase (SA-β-Gal) activity, a marker of senescence, was significantly increased in the vein and artery in the AVF-CKD model at one week (FIG. 5) and at two weeks (FIG. 6), after the creation of the fistula.

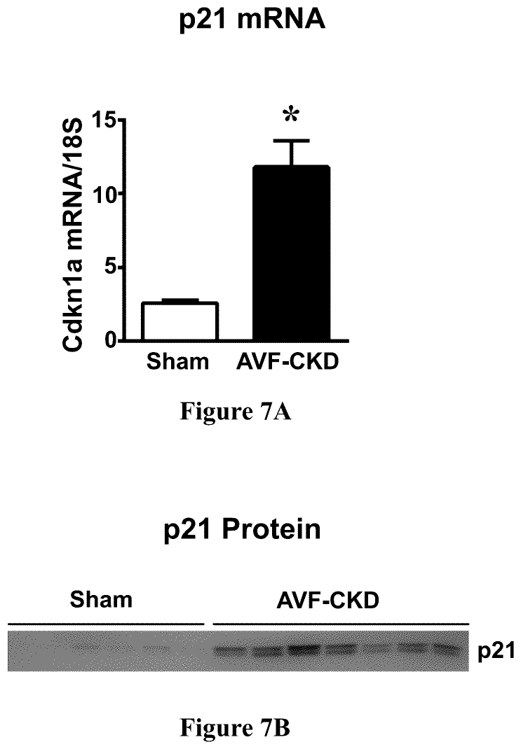

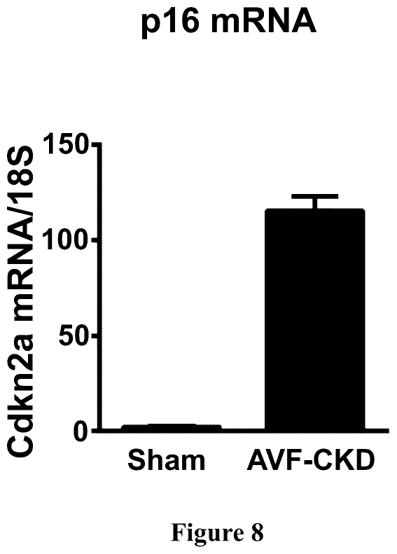

At one week, there was evidence of marked induction of p21Cip1, as reflected both by expression of the p21Cip1 (Cdkn1a) mRNA (FIG. 7A) and the p21 polypeptide (FIG. 7B). There also was an induction of p16Ink4a as reflected by expression of Cdkn2a mRNA (FIG. 8).

These results demonstrate that the senescence phenotype observed in the fistula in mice (see, e.g., Example 1) also is observed in rats.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-18. (canceled)

19. A method for maintaining functionality of venous graft within a mammal, wherein said method comprises administering a composition comprising a senolytic agent to said mammal, wherein said venous graft remains functional as effective graft following said administering step.

20. The method of claim 19, wherein said mammal is a human.

21. The method of claim 19, wherein said composition comprises dasatinib, a tocotrienol, quercetin, or a combination thereof.

22. The method of claim 19, wherein said composition comprises dasatinib and quercetin.

23. The method of claim 19, wherein said composition comprises a tocotrienol and quercetin.

24. The method of claim 19, wherein said administration comprises a local administration to said venous graft.

25. The method of claim 19, wherein said venous graft bypasses an occluded artery within said mammal.

26. The method of claim 19, wherein said venous graft is attached to a coronary artery.

27. The method of claim 26, wherein said mammal has an ischemic heart disease.

28. The method of claim 19, wherein said venous graft is attached to a carotid artery.

29. The method of claim 28, wherein said mammal has a cerebrovascular disease.

30. The method of claim 19, wherein said venous graft is attached to a femoral or popliteal artery.

31. The method of claim 30, wherein said mammal has a peripheral arterial disease.

32. The method of claim 19, wherein said venous graft is attached to a renal artery.

33. The method of claim 31, wherein said mammal has a renovascular disease.

34. The method of claim 19, wherein said venous graft is attached to a mesenteric artery.

35. The method of claim 34, wherein said mammal has a mesenteric arterial disease or abdominal angina.
